(19) United States
(12) Patent Application Publication
(10) Pub. No.: US 2005/0187430 A1
(43) Pub. Date: Aug. 25, 2005

(54) Fluid charging mechanism for balloon catheter system

(75) Inventors: Knud T. Aundal, Roskilde (DK); Anders Pedersen, Herlev (DK); Therese Levin, Viken (SE); Marianne Nielsen, Helsinge (DK)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/782,652

(22) Filed: Feb. 19, 2004

(51) Int. Cl. A61F 2/02

(52) U.S. Cl. 600/31; 606/192

(57) ABSTRACT

In general, the invention is directed to a balloon catheter system for intra-luminal or intra-cavity pressure measurements. In accordance with the invention, the balloon catheter system provides a closed fluid charging mechanism to charge and discharge a balloon mounted on the catheter. More particularly, the charging mechanism may include a closed reservoir, a passage, and an actuator that acts to drive fluid out of the reservoir and through the passage. A lumen within a catheter may transport fluid from the passage to charge the balloon. The pressure monitoring catheter system may take pressure measurements when the balloon is charged. Discharging the balloon may include withdrawing the actuator from the charging mechanism, which fills the reservoir with fluid from the balloon. In addition, a seal member may be used in the pressure monitoring catheter system to provide a more sealed environment for holding and transporting fluid.

### TECHNICAL FIELD

The invention relates to catheter systems and, more particularly, balloon catheter systems for intra-luminal or intra-cavity pressure measurement.

### BACKGROUND

In general, a catheter is a hollow flexible tube for insertion into a body cavity, duct, or vessel to allow the passage of fluids or to dilate a passageway. Catheters are often used for delivering diagnostic and therapeutic agents to internal target sites that may be accessed via the circulatory system, the urinary system, or the digestive system.

Some catheter systems are used for intra-luminal and intra-cavity diagnostic purposes, such as gastrointestinal, intracranial, intrauterine, and vesicle evaluations. As an example, some catheter systems may incorporate pressure transducers for urodynamic pressure evaluation for the diagnosis of incontinence or other urinary tract disorders.

Structurally, a pressure measurement catheter system may include a balloon mounted on a catheter shaft. The balloon may be charged with a fluid, such as gas or liquid, to inflate the balloon to a desired diameter, volume, and/or pressure. Charging the balloon involves introduction of air or liquid from an external port that draws the fluid from the environment outside of the catheter. In this sense, existing catheter systems incorporate an open charging mechanism, i.e., a charging mechanism that relies on introduction of fluid from the environment outside of the catheter.

When the balloon is charged, a pressure transducer in fluid communication with the interior of the balloon measures pressure levels or changes. A pressure-monitoring device analyzes the measured pressure to aid in diagnosis. Upon completion of the desired pressure measurements, the balloon is discharged, typically by venting the charging fluid from the balloon via an external port to the environment outside the catheter.

### SUMMARY

In general, the invention is directed to a balloon catheter system for intra-luminal or intra-cavity pressure measurements. In accordance with the invention, the balloon catheter system provides a closed fluid charging mechanism to charge and discharge a balloon mounted on the catheter. The invention has certain objects. That is, various embodiments of the present invention provide solutions to one or more problems existing in the prior art with respect to existing balloon catheter systems for pressure measurement.

The problems include, for example, the risk of over-inflation or under-inflation of the balloon mounted on the catheter due to imprecision in the amount of fluid introduced into the balloon from an external port. Over-inflation or under-inflation can undermine the accuracy of pressure measurements. Another problem is the possibility of leakage of fluid from the balloon to the outside environment, causing variation in the baseline pressure within the balloon. In addition, an external port, and the internal charging and pressure lumens in communication with the external port, can be susceptible to blockage due to introduction of debris from the outside environment. There is also a risk of contamination of the catheter, and transmission of the contamination to the patient, by introduction of debris or biological agents from the outside environment. As a further problem, the pressure sensor, in fluid communication with the charging port and charging lumens, can also be susceptible to contamination and require cleaning.

Various embodiments of the present invention have the object of solving the foregoing problems. For example, it is an object of the present invention to reduce the risk of over-inflation or under-inflation of the balloon, and instead provide a precise volume of fluid to the balloon to establish a stable baseline pressure. In addition, it is an object to reduce the possibility of leakage of the fluid from the balloon, thereby maintaining the charge on the balloon during the course of the pressure measurement. It is a further object of the invention to prevent blockage and contamination within the charging and pressure measurement lumens of the balloon catheter, and thereby ensure proper charging, avoid pressure fluctuations, reduce the risk of patient contamination, and reduce the need to clean the pressure sensor, as well as potential corrosion and damage to parts.

Various embodiments of the invention may possess one or more features capable of fulfilling the above objects. In general, the invention includes a sensor body, a charging mechanism, a catheter body, a catheter extending from the catheter body, and a balloon mounted on the catheter. The charging mechanism includes a closed reservoir containing a volume of fluid, which may be either a gas or a liquid. A passage couples the reservoir to a lumen that extends with the catheter to the balloon.

An actuator in the charging mechanism drives the fluid out of the reservoir and through the passage to charge the balloon. The actuator may include a piston, a plunger, a plastic nipple, a screw, or any other device that may drive fluid from the reservoir. The sensor body is coupled to the catheter body and includes a lumen in fluid communication with the lumen extending within the catheter.

A pressure sensor in the sensor body measures pressure levels or changes when the balloon is charged. To discharge the balloon, fluid is withdrawn to refill the closed reservoir. For example, the actuator may be used to create a negative pressure that draws the fluid back into the reservoir, thereby deflating the balloon for removal of the catheter from a body lumen or cavity. Hence, the pressure monitoring catheter system may use the same fluid to charge and discharge the balloon. The closed reservoir is substantially sealed from the environment outside the catheter.

In one embodiment, the invention provides a catheter system comprising a catheter defining an internal lumen, a balloon mounted on the catheter, the balloon defining an internal chamber in fluid communication with the lumen, a charging mechanism including a closed reservoir, a passage, and an actuator to charge and discharge the balloon with at least a portion of a volume of fluid contained in the closed reservoir, wherein the closed reservoir is in fluid communication with the lumen via the passage and substantially sealed from an environment outside the catheter system, and a pressure sensor in fluid communication with the lumen to sense a pressure of the fluid.

In another embodiment, the invention provides a method comprising placing a catheter into a patient, wherein the catheter includes an internal lumen and a balloon defining an interior chamber in fluid communication with the lumen, sensing a first pressure within the lumen, charging the balloon with at least a portion of a volume of fluid contained within a closed reservoir, and sensing a second pressure within the lumen.

In a further embodiment, the invention provides a catheter system comprising a catheter defining an internal lumen, a balloon mounted on the catheter, the balloon defining an internal chamber in fluid communication with the lumen, means for charging the balloon with at least a portion of a volume of fluid contained within a closed reservoir, wherein the reservoir is substantially sealed from an environment outside the catheter system, and means for sensing a pressure of the fluid.

In another embodiment, the invention provides a catheter body for a balloon catheter system, the catheter body comprising a first fitting to couple the catheter body to a catheter, a second fitting to couple the catheter body to a sensor body, a charging mechanism including a closed reservoir, a passage, and an actuator to charge and discharge a balloon mounted on the catheter via a lumen with at least a portion of a volume of fluid contained in the closed reservoir, wherein the closed reservoir is in fluid communication with the lumen via the passage and substantially sealed from an environment outside the balloon catheter system.

In an added embodiment, the invention provides a sensor body for a balloon catheter system, the sensor body comprising a first fitting to couple the sensor body to a catheter body, a second fitting to couple the sensor body to a monitor, a charging mechanism including a closed reservoir, a passage, and an actuator to charge and discharge a balloon mounted on the catheter via a lumen with at least a portion of a volume of fluid contained in the closed reservoir, wherein the closed reservoir is in fluid communication with the lumen via the passage and substantially sealed from an environment outside the balloon catheter system.

In a further embodiment, the invention provides an intermediate charging body for a balloon catheter system, the charging body comprising a first fitting to couple the charging body to a catheter body coupled to a balloon catheter, a second fitting to couple the charging body to a sensor body, a charging mechanism including a closed reservoir, a passage, and an actuator to charge and discharge a balloon mounted on the catheter via a lumen with at least a portion of a volume of fluid contained in the closed reservoir, wherein the closed reservoir is in fluid communication with the lumen via the passage and substantially sealed from an environment outside the balloon catheter system.

In comparison to known implementations of balloon catheter pressure measurement systems, various embodiments of the present invention may provide one or more advantages. For example, various embodiments of the present may reduce the risk of over-inflation or under-inflation of the balloon, as well as the risk of leakage, by providing a closed reservoir containing a precise volume of fluid. In addition, the closed reservoir of the balloon catheter system of the invention serves to prevent blockage and contamination within the charging and pressure measurement lumens of the balloon catheter, thereby ensuring proper charging, avoiding pressure fluctuations, reducing the risk of patient contamination, and reducing the need to clean the pressure sensor. In addition, the balloon catheter system may actively discharge the fluid from the balloon when the actuator returns to its initial position, thereby emptying the balloon, and facilitating safe removal of the catheter from the patient.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A through 1C are block diagrams illustrating various exemplary forms of a balloon catheter system for pressure measurement.

FIGS. 2A through 2E are schematic diagrams illustrating various embodiments of a balloon catheter system for pressure measurement in accordance with the invention.

FIGS. 3A through 3E are schematic diagrams illustrating operation of a balloon catheter system in accordance with the invention.

FIGS. 4A through 4G are schematic diagrams illustrating seal members for use in coupling various elements of a pressure monitoring catheter system in accordance with the invention.

FIGS. 5A through 5E are schematic diagrams illustrating alternative seal members for use in coupling various elements of a pressure monitoring catheter system in accordance with the invention.

FIGS. 6A through 6E are schematic diagrams illustrating more alternative seal members for use in coupling various elements of a pressure monitoring catheter system in accordance with the invention.

FIG. 7 is a flow diagram illustrating operation of a pressure monitoring balloon catheter system in accordance with the invention.

### DETAILED DESCRIPTION

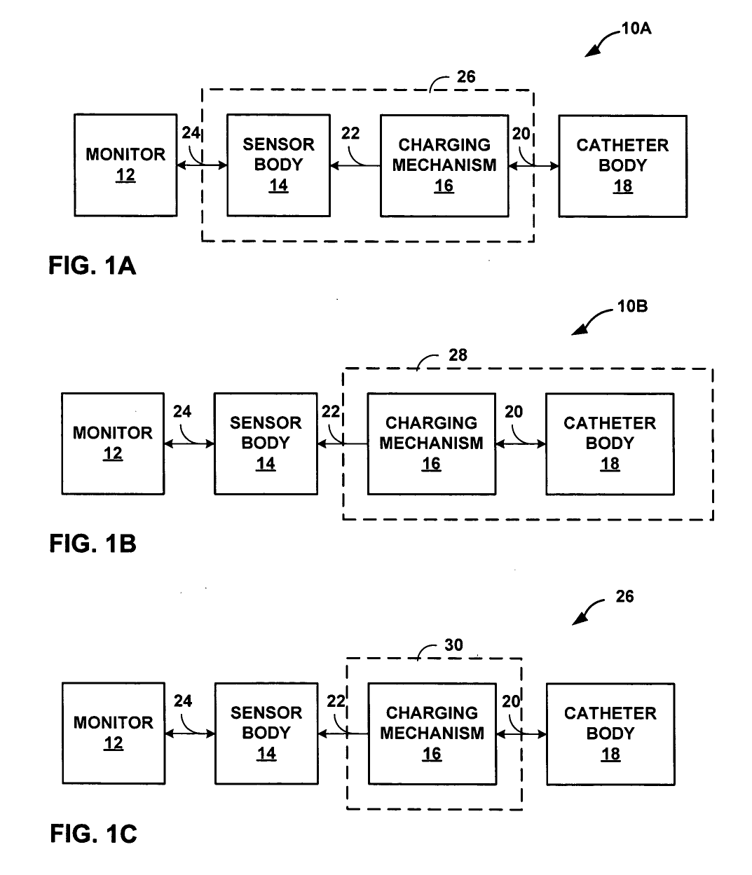

FIGS. 1A through 1C are block diagrams illustrating various exemplary forms of a balloon catheter system 10 for pressure measurement. In general, balloon catheter system 10 includes a monitor 12, a sensor body 14, a charging mechanism 16 and a catheter body 18. As will be described, a catheter extends from catheter body, 18, and a balloon is mounted on the catheter. In addition, charging mechanism 16 includes a reservoir containing a volume of fluid, which may be either a gas or a liquid. The fluid may include a gas such as atmospheric air, aspired from the environment. Alternative fluids may include carbon dioxide, saline, or water. For some embodiments, e.g., for urodynamic applications, the reservoir volume may be less than the volume of a balloon. In this manner, the balloon may be only partially filled and not overinflated so as to avoid excessive wall tension from the balloon that interferes with pressure measurements. For example, the maximum volume of the balloon may range from approximately 30 μl to approximately 40 μl. In addition, the volume of the reservoir may range from approximately 15 μl to approximately 25 μl. The balloon may be filled with fluid to 50-80% of its maximum volume. The reservoir is substantially sealed from the environment outside the catheter, making it a “closed” reservoir. A passage couples the reservoir to a lumen 20 that extends within the catheter to the balloon. An actuator in charging mechanism 18 drives the fluid out of the reservoir and through the passage to charge the balloon.

Sensor body 14 is coupled to the catheter body 18 via charging mechanism 16 and to a lumen 22 in fluid communication with lumen 20 extending within the catheter. A pressure sensor in sensor body 14 measures pressure levels or changes when the balloon is charged via lumens 20, 22. To discharge the balloon, fluid is withdrawn to refill the closed reservoir. For example, the actuator within charging mechanism 16 may be used to create a negative pressure that draws the fluid back into the reservoir, thereby deflating the balloon for removal of the catheter from a body lumen or cavity. Hence, pressure monitoring catheter system 10 may use the same fluid to charge and discharge the balloon. An electrical or optical cable 24 connects the sensor within sensor body 14 to monitor 12.

In the example of FIG. 1A, a balloon catheter system 10A represents integration of charging mechanism 16 with sensor body 14 to form an integrated sensor/charging element 26. Combining the sensor body 14 and charging mechanism 16 into one component may simplify the medical procedure. For example, this combination reduces the number of components of system 10A. In addition, with elimination of charging mechanism 16, catheter body 18 may be less expensive and thus disposable.

In the example of FIG. 1B, a balloon catheter system 10B represents integration of charging mechanism 16 with catheter body 18 to form an integrated catheter/charging element 28. Combining catheter body 18 and charging mechanism 16 into one component may simplify a medical procedure, reducing the number of components of system 10B.

In the example of FIG. 1C, a balloon catheter system 10C represents incorporation of a dedicated charging mechanism as an intermediate coupling element 30 disposed between sensor body 14 and catheter body 18. In this embodiment, charging mechanism 16 may be maintained independently of catheter body 18 and sensor body 14, and may permit use with a variety of different catheter bodies and sensor bodies.

FIGS. 1A-1C illustrate how different components of an exemplary balloon catheter system 10 may fit together according to various embodiments of the invention. In operation, charging mechanism 16 comprises a controlled volume of fluid from the closed reservoir. The reservoir does not rely on an external port of vent the environment outside catheter system 10. Instead, the reservoir contains a volume of fluid that is substantially sealed from the outside environment outside the catheter, providing a “closed” balloon catheter system. Pressure sensor of sensor body 14 senses a pressure of the fluid within lumen 20, and communicates a signal representative of the sensed pressure to a monitor 12 via a cable 24.

Monitor 12 processes the sensed pressure signal to produce a pressure measurement. The pressure measurement may be a pressure level, pressure change, pressure trend, or some other pressure-related parameter helpful in diagnosis of patient physiological condition. For example, a clinician may use the balloon catheter system 10 to sense a variety of physiological parameters such as bladder pressure, urethra pressure, blood pressure, intracranial pressure, intrauterine pressure, pulmonary artery pressure, and other intra-luminal or intra-cavity pressures.

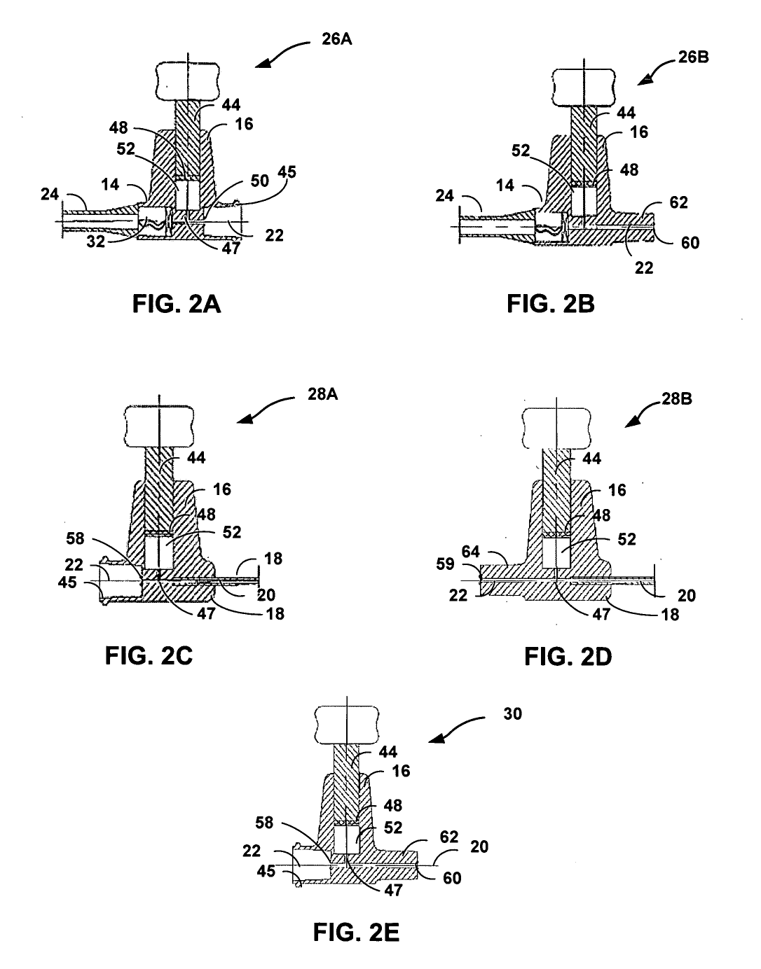

FIGS. 2A through 2E are schematic diagrams illustrating various embodiments of a balloon catheter system 10 for pressure measurement in accordance with the invention. In particular, FIGS. 2A through 2E depict exemplary structures for sensor body 14, charging mechanism 16 and catheter body 18.

FIG. 2A shows a combined sensor/charging element 26A, as in FIG. 1A, in more detail. Sensor/charging element 26A incorporates both charging mechanism 16 and a sensor body 14. In addition, sensor body 14 includes a pressure sensor 32, which may take a variety of forms, such as a strain gauge, piezoelectric transducer, or the like. Charging mechanism 16 includes a closed reservoir 52 containing a volume of fluid, a passage 47, and an actuator 44 that acts to drive fluid out of the reservoir and into lumen 22 via passage 47. In particular, actuator 44 may take the form of a piston, a plunger, a plug, a screw, or any other structure suitable to drive at least a portion of fluid from the reservoir to lumen 22 for introduction into lumen 20 defined by the catheter and catheter body 18. In the example of FIG. 2A, actuator 44 is a piston that is forced downward into reservoir 52 to drive fluid out of the reservoir.

A fluid seal may be formed between the distal end of actuator 44 and reservoir 52. The fluid seal may be formed using seal member 48. The fluid seal may include a deformable surface that deforms under compression upon engagement of the distal end of actuator 44 and an interior wall of reservoir 52. In addition, in some embodiments, upon full extension of actuator 44 into reservoir 52, fluid seal member 48 may seal off passage 47 to prevent backflow of fluid into reservoir 52.

Cable 24 extends from one end of sensor/charging element 26A sensor 32 to provide connectivity between sensor 32 and monitor 12. Sensor/charging element 26A defines, at an opposite end, a female fitting 45 designed to receive a reciprocal male fitting from catheter body 18. A seal member 50 may be provided within female fitting 45 to form a fluid seal between sensor body 20 and catheter body 18. Like seal member 48, seal member 50 may include a deformable surface that deforms under compression upon engagement of a male fitting of catheter body 24 and the female fitting of sensor/charging element 26A.

FIG. 2B shows a combined sensor/charging element 26B similar to sensor/charging element 26A, with the difference being in the way sensor/charging element 26B is able to connect with a catheter body 18. In particular, sensor/charging element 26B has a male fitting 62 that may be used with a reciprocal female fitting presented by catheter body 18. Seal member 60 may be provided within female fitting 62 to form a fluid seal between sensor/charging element 26B and a compatible catheter body 18.

FIG. 2C shows a combined catheter/charging element 28A, as in FIG. 11B, in more detail. In the example of FIG. 2C, charging mechanism 16 is included within catheter body 18 to form catheter/charging element 28A. Catheter/charging element 28A may also include a female fitting 59 at another end for compatibility with a male fitting presented by a sensor body 14. Seal member 58 may provide a fluid seal between catheter body 24 and a compatible sensor body. Lumen 22 may be coupled to the compatible sensor body for pressure sensing. %

FIG. 2D shows a combined catheter/charging element 28B similar to catheter/charging element 28A from FIG. 2C, with the difference being in the way catheter/charging element 28B is able to connect with sensor body 14. In particular, catheter body 18 has a male fitting 64 that may be used with a reciprocal female fitting presented by sensor body 14. Seal member 59 may be provided within male fitting 64 to form a fluid seal between catheter/charging element 28B and a compatible sensor body 14.

FIG. 2E shows an intermediate element 30 that includes charging mechanism 16, a female fitting 45 that may be used with a reciprocal male sensor body 14, and a male fitting 62 that may be used with a reciprocal female catheter body 18. Alternatively, intermediate element 30 may comprise charging mechanism 16, a male fitting that may be used with a reciprocal female sensor body 14, and a female fitting that may be used with a reciprocal male catheter body 18. Seal member 58 may be provided within female fitting 45 to form a fluid seal between intermediate element 30 and compatible sensor body 14. In addition, seal member 60 may be provided within male fitting 62 to form a fluid seal between intermediate element 30 and catheter body 18. FIG. 2E shows seal member 58 and seal member 60 that may be used as described above.

The catheter systems illustrated by FIG. 2 may have alternative implementations than those that are shown. For example, actuator 44 may take the form of a plunger, a plug, a screw, or any other structure suitable to drive at least a portion of fluid from the reservoir to lumen 22 for introduction into lumen 20 defined by the catheter and catheter body 18

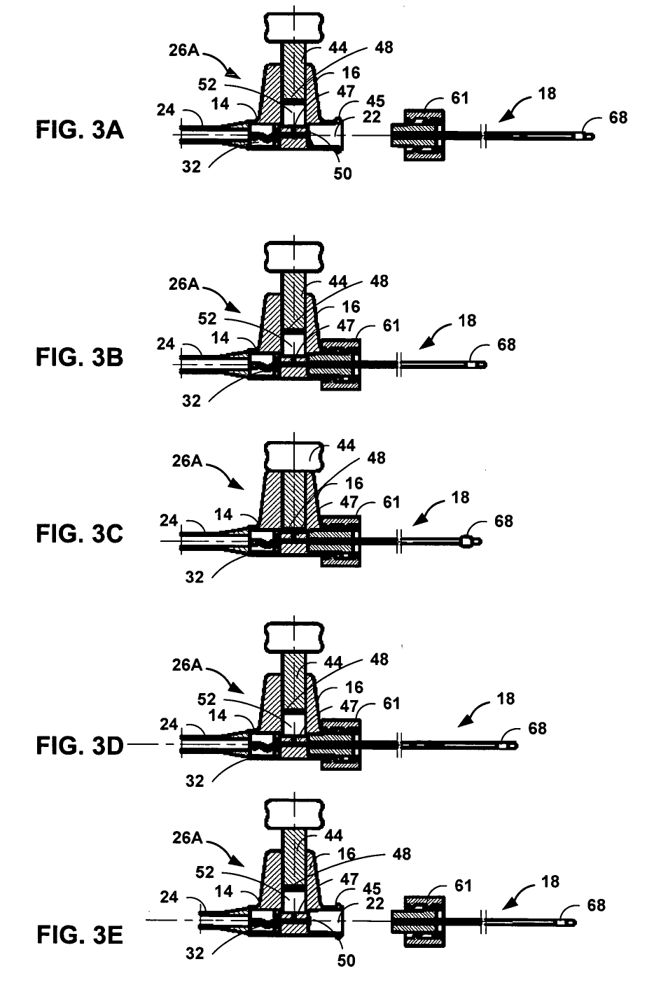

FIGS. 3A through 3E are schematic diagrams illustrating operation of a balloon catheter system in accordance with the invention. In particular, FIGS. 3A through 3E illustrate a sequence of events that may occur when charging and discharging a balloon 68 in the balloon catheter system.

FIG. 3A shows a combined sensor/charging element 26A, from FIG. 2A, along with male catheter body 18. As illustrated, balloon 68 may be mounted to catheter body 18. In addition, a male fitting 61 may be attached to catheter body 18 for use with a reciprocal female fitting 45 on sensor/charging element 26A, which incorporates both charging mechanism 16 and a sensor body 14. In addition, sensor body 14 includes a pressure sensor 32, which may take a variety of forms, such as a strain gauge, piezoelectric transducer, or the like.

Charging mechanism 16 includes a closed reservoir 52 containing a volume of fluid, a passage 47, and an actuator 44 that acts to drive fluid out of the reservoir and into lumen 22 via passage 47. In particular, actuator 44 may take the form of a piston, a plunger, a plug, a screw, or any other structure suitable to drive at least a portion of fluid from the reservoir to lumen 22 for introduction into lumen 20 defined by the catheter and catheter body 18. In the example of FIG. 3A, actuator 44 is a piston that is forced downward into reservoir 52 to drive fluid out of the reservoir.

A fluid seal may be formed between the distal end of actuator 44 and reservoir 52. The fluid seal may be formed using seal member 48. The fluid seal may include a deformable surface that deforms under compression upon engagement of the distal end of actuator 44 and an interior wall of reservoir 52. In addition, in some embodiments, upon full extension of actuator 44 into reservoir 52, fluid seal may seal off passage 47 to prevent backflow of fluid into reservoir 52.

Cable 24 extends from one end of sensor/charging element 26A sensor 32 to provide connectivity between sensor 32 and monitor 12. Sensor/charging element 26A defines, at an opposite end, female fitting 45 designed to receive a reciprocal male fitting from catheter body 18. A seal member 50 may be provided within female fitting 45 to form a fluid seal between sensor body 20 and catheter body 18. Like seal member 48, seal member 50 may include a deformable surface that deforms under compression upon engagement of a male fitting of catheter body 24 and the female fitting of sensor/charging element 26A.

FIG. 3B shows sensor/charging element and catheter body 18 fitting together, as necessary for charging balloon 68. The catheter body 18 may be placed in the patient as dictated by the needs of a particular medical or diagnostic procedure. In addition, seal member 50 (shown in FIG. 3A) may create a fluid seal between sensor/charging element 26A and catheter body 18. The fluid seal may include a deformable surface that deforms under compression upon engagement of the sensor/charging element 26A and catheter body 18.

FIG. 3C shows actuator 44, illustrated in FIG. 3 as a piston, charging balloon 68 with at least a portion of the volume of fluid in the reservoir. Alternatively, charging the balloon 68 may comprise pushing on an elastic nipple, moving an alternative actuator into the closed reservoir, or any technique that includes driving at least a portion of fluid from the reservoir 52 to balloon 68. In the exemplary technique of FIG. 3, actuator 44 may be pushed downward onto an inner wall of charging mechanism 16, which moves some of the fluid in the reservoir 52 to charge balloon 68.

In addition, seal member 48 may create a fluid seal between the distal end of actuator 44 and reservoir 52. The fluid seal may include a deformable surface that deforms under compression upon engagement of the distal end of actuator 44 and an interior wall of reservoir 52. In addition, in some embodiments, upon full extension of actuator 44 into reservoir 52, fluid seal may seal off passage 47 to prevent backflow of fluid into reservoir 52.

In one embodiment, a plurality of balloons may be mounted axially and/or radially to catheter body 18. The balloons may be charged individually by a number of charging mechanisms using a technique similar to the technique described for use with only one balloon 68, wherein each charging mechanism corresponds to an individual balloon.

In any event, while balloon 68 is charged, a pressure measurement may be taken. The pressure measurement may be a pressure level, pressure change, pressure trend, or some other pressure-related parameter helpful in diagnosis of patient physiological condition. For example, a clinician may use sensor body 14 to sense a variety of physiological parameters such as bladder pressure, urethra pressure, blood pressure, intracranial pressure, intrauterine pressure, pulmonary artery pressure, and other intra-luminal or intra-cavity pressures. Monitor 12, which may be coupled to cable 24, may process the sensed pressure signal to produce a pressure measurement.

FIG. 3D shows actuator 44 discharging balloon 68 by moving at least a portion of the volume of fluid from balloon 68 to reservoir 52. In particular, the figure shows actuator 44 moving out of the closed reservoir, which uses negative pressure to allow fluid to move from balloon 68 into reservoir 52. This action may move some of the fluid in balloon 68 to deflate the balloon 68. In particular, because the catheter system is closed, the same fluid used to charge the balloon may be used to discharge the balloon. In another embodiment, discharging balloon 68 may comprise detaching the catheter body 18 from sensor body 14. Alternatively, discharging balloon 68 may involve venting the fluid from the balloon to an outside environment, or any other technique that moves fluid out of balloon 68.

FIG. 3E shows catheter body 18 decoupled from sensor/charging element 26A upon completion of the pressure measuring operation. In one embodiment, disconnecting sensor/charging element 26A may be a technique for discharging balloon 68. Disconnecting catheter body 18 from sensor body 20 may allow fluid from catheter body 18 to vent out of catheter body 18. In one embodiment, catheter body 18 may be thrown away after being used in a single operation.

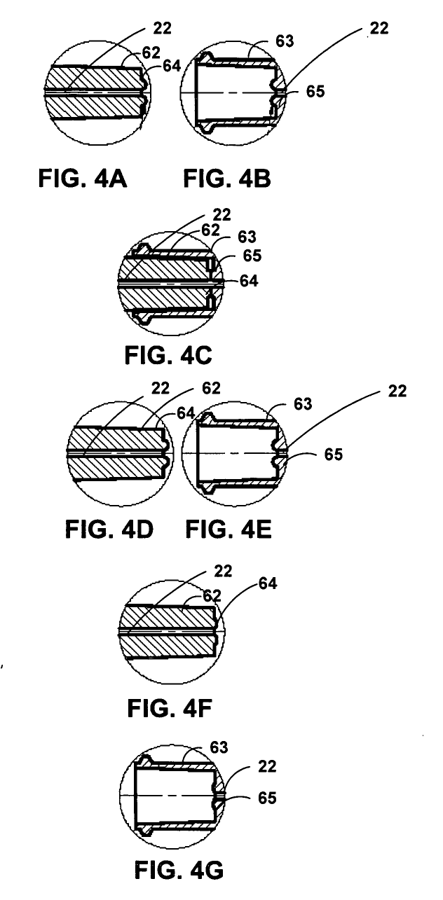

FIGS. 4-6 illustrate possible implementations of seal members that may be used between elements of a pressure monitoring catheter system to seal off lumen 22, or other lumens or channels. The seal members may be ring-like members that are integrated with or mounted to particular fittings. The seal members may be generally concentric with the lumens. In particular, the seal members illustrated may be used to form a fluid seal between a sensor body 14 and an intermediate body 30, an intermediate body 30 and a catheter body 18, a sensor/charging element 26 (26A or 26B) and a catheter body 18, or between a sensor body 14 and a catheter/charging element 28 (28A or 28B). Intermediate body 30 may include a charging mechanism 16, but no sensor body 14 or catheter body 18. The seal members described in FIGS. 4-6 may include seal members 48, 50, 58, 59, 60, or other seal members that may be used with the balloon catheter system.

The discussion of FIGS. 4-6 will discuss male and female fittings without specifying whether they belong to a sensor/charging element 26 (26A or 26B), a catheter/charging element 28 (28A or 28B), a sensor body 14, an intermediate body 30, or a catheter body 18. It is understood that each of a sensor/charging element 26 (26A or 26B), a catheter/charging element 28 (28A or 28B), a sensor body 14, an intermediate body 30, and a catheter body 18 may comprise a male or female fitting that may be coupled to a compatible male or female fitting. Therefore, the discussion may proceed by describing male and female fittings generically without specifically calling out the fitting with which they are associated.

FIGS. 4A through 4G are schematic diagrams illustrating seal members for use in coupling various elements of a pressure monitoring catheter system in accordance with the invention. In particular, FIGS. 4A through 4G illustrate an implementation of a seal, wherein a seal member is used on both a male and female fitting. FIGS. 4A and 4B show a male fitting 62 and female fitting 63 comprising lumen 22. Male fitting 62 and female fitting 63 may include seal members 64 and 65 respectively. In one embodiment, a seal member may be made of the same material as the male or female component to which it is coupled. In another embodiment, a seal member may simply be part of the male or female fittings. Male fitting 62 and female fitting 63 may be fit together as in FIG. 4C. The seal members 64 and 65 may form a fluid seal between the male fitting 62 and female fitting 63 so that fluid may pass between the male and female fittings without substantial leakage.

FIGS. 4D and 4E show seal members 64 and 65, respectively, after they have been disconnected. Elastic properties prevent continued deformation of seal members 64 and 65 after the seal members are disengaged. In one embodiment, however, a one of seal members 64 and 65 may be deformed during and after engagement. FIG. 4F shows seal member 64 deformed after seal members 64 and 65 are disengaged. FIG. 4G shows seal member 65 deformed after seal members 64 and 65 are disengaged.

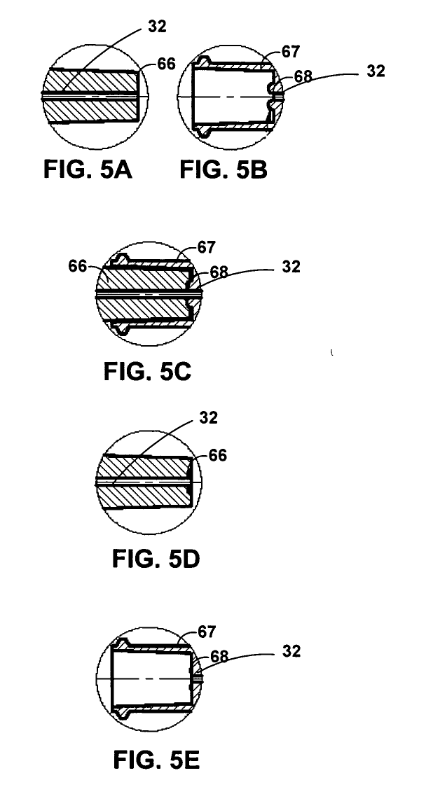

FIGS. 5A through 5G are schematic diagrams illustrating seal members for use in coupling various elements of a pressure monitoring catheter system in accordance with the invention. In particular, FIG. 5 illustrates an alternative implementation of a seal, wherein only one seal member is used. In particular, a seal member may be used on a female fitting but not on a male fitting.

FIGS. 5A and 5B show a male fitting 66 and female fitting 67 comprising lumen 22. In one embodiment, seal member 68 may be made of the same material as the female component to which it is coupled. In another embodiment, a seal member may simply be part of the female fitting. Male fitting 66 and female fitting 67 may be fit together as in FIG. 5C. The seal member 68 may form a fluid seal between the male and female fittings so that fluid may pass between the male and female fittings without substantial leakage.

FIG. 5D shows male fitting 66 after male fitting 66 and female fitting 67 are disengaged. In one embodiment, seal member 68 may be deformed during and after engagement. FIG. 5E shows female fitting 67 deformed after male fitting 66 and female fitting 67 are disengaged. In one embodiment, seal member 68 may be made of elastic material, as is seal member 65 from FIG. 4B.

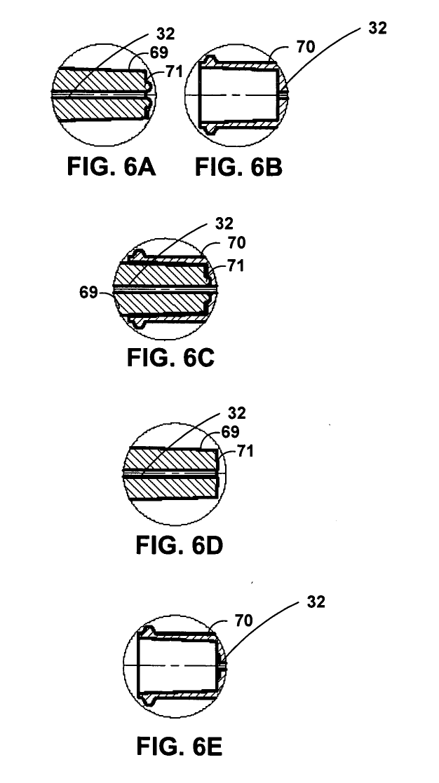

FIGS. 6A through 6E are schematic diagrams illustrating more alternative seal members for use in coupling various elements of a pressure monitoring catheter system in accordance with the invention.

In particular, a seal member is used on a male fitting but not on a female fitting. FIGS. 6A and 6B show a male fitting 69 and female fitting 70 comprising lumen 22. In one embodiment, seal member 71 may be made of the same material as the male component to which it is coupled. In another embodiment, a seal member may simply be an integrated part of the male fitting. For example, the seal member may be integrally molded with the male fitting. Alternatively, the seal member may be adhesively bonded or ultrasonically welded to the male fitting. Male fitting 69 and female fitting 70 may be fitted together as in FIG. 6C. The seal members 64 and 65 may form a fluid seal between the male and female fittings so that fluid may pass between the male and female fittings without substantial leakage.

FIG. 6D shows male fitting 69 after the male fitting and female fitting 70 are disengaged. In one embodiment, seal member 71 may be deformed during and after engagement. Use of deformable materials may ensure single use of the device. Seal member 71 may comprise deformable plastic materials such as polypropylene, polyethylene, acetyl butyl styrene, polyvinyl sulfane, polyoxymethylene, or the like. FIG. 6E shows female fitting 70 deformed after male fitting 69 and female fitting 70 are disengaged. In one embodiment, seal member 71 may be made of elastic material, as is seal member 65 from FIG. 4B. The elastic material may comprise natural rubber, silicone, or the like. Alternatively, seal member 71 may comprise non-deformable materials such as hard plastics, metals such as aluminum and steel, and ceramics such as glass and porcelain.

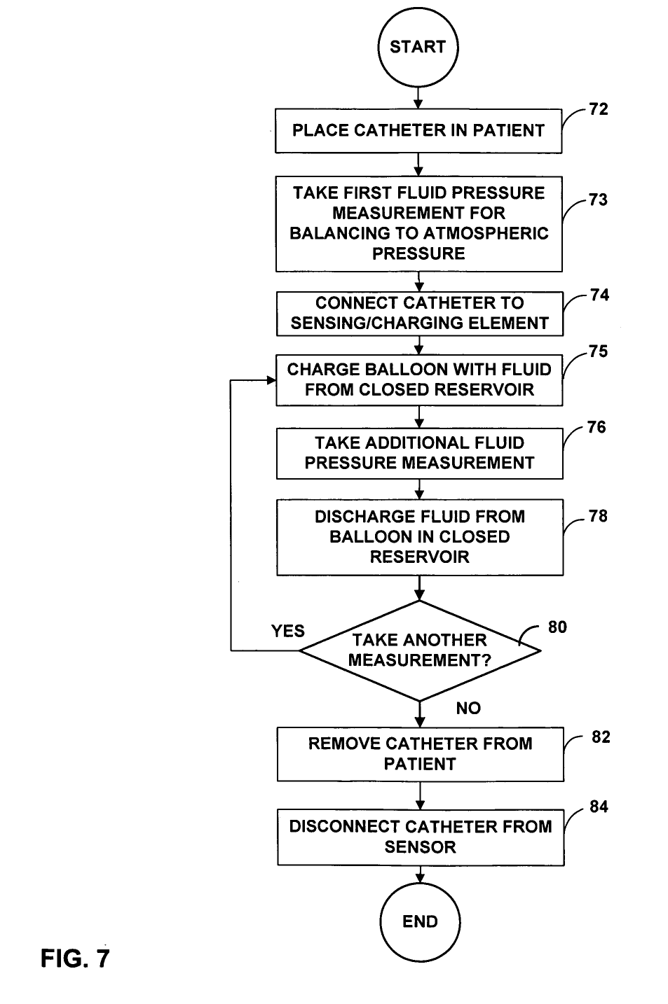

FIG. 7 is a flow diagram illustrating operation of a pressure monitoring balloon catheter system in accordance with the invention. For purposes of explanation, an embodiment with a sensor/charging element 26A will be discussed. In particular, a catheter body 18, defining a lumen 22, may be in fluid communication with a sensor/charging element 26A for charging a balloon 68 with a volume of fluid. A charging mechanism 16 of sensor/charging element 26A may be coupled to the lumen 22 of the catheter body 18.

Charging mechanism 16 may include a reservoir 52 containing a volume of fluid, wherein the reservoir is in fluid communication with lumen 22 and substantially sealed from an environment outside the catheter system. A sensor body 14 may include a pressure sensor 32 in fluid communication with lumen 22 to sense a pressure of the fluid. The catheter body 18 and sensor/charging element 26A may include male or female fittings that are compatible for forming a connection. For example, catheter body 18 may comprise a male fitting that fits into a female fitting of the sensor/charging element 26A. The connection may include a seal member 50 to create a fluid seal between sensor/charging element 26A and catheter body 18. The fluid seal may prevent substantial leakage of fluid.

Initially, catheter body 18 with a balloon 68 mounted to it may be placed in a patient (72) in preparing to sense a variety of physiological parameters such as bladder pressure, urethra pressure, blood pressure, intracranial pressure, intrauterine pressure, pulmonary artery pressure, and other intra-luminal or intra-cavity pressures.

A first fluid pressure measurement may be taken (73) by sensor body 14. This measurement may include calibration so further fluid pressure measurements may have meaning relative to the first fluid pressure measurement. In particular the first measurement may aid in balancing the fluid pressure to the atmospheric pressure. In one embodiment, a monitor 12 may analyze the measurement for evaluation purposes.

In one embodiment, catheter body 18 may be connected to charging mechanism 16 from sensor/charging element 26A (74). The charging mechanism 16 may charge the balloon 68 with at least a portion of the volume of fluid in the reservoir 52 by moving an actuator 44 into the reservoir (75). The actuator may include a piston, a plunger, a plug, a screw or any other device that may drive fluid from the reservoir to the balloon. Alternatively, the balloon may be charged by pressing on an elastic nipple to move fluid into the balloon.

Upon charging the balloon 68, a second fluid pressure measurement may be taken (76). The sensor body 20 may feed the second measurement to the monitor 12, which may analyze the second measurement. Monitor 12 processes the sensed pressure signal to produce a pressure measurement. The pressure measurement may be a pressure level, pressure change, pressure trend, or some other pressure-related parameter helpful in diagnosis of patient physiological condition. For example, a clinician may use the balloon catheter system 10 to sense a variety of physiological parameters such as bladder pressure, urethra pressure, blood pressure, intracranial pressure, intrauterine pressure, pulmonary artery pressure, and other intra-luminal or intra-cavity pressures.

After the second pressure measurement is taken, charging mechanism 16 may discharge the fluid from the balloon (78). Discharging the fluid from the balloon 68 may include withdrawing actuator 44 from the closed reservoir, which may allow fluid from balloon 68 to return to the closed reservoir 52. Alternatively, discharging the balloon may comprise disconnecting the catheter body from the sensor/charging element 26A. The balloon may be charged (75) again if another measurement is desired (80). In other words, balloon 68 may be recharged and an additional measurement may be taken before the balloon is discharged again.

When no more measurements are desired, catheter body 18 may be removed from the patient (82), and the catheter body may be disconnected from the sensor body (84) if it has not been disconnected already.

The description of the flow diagram focused on the embodiment with sensor/charging element 26A. These and other embodiments are within the scope of the following claims. For example, a catheter/charging element may include charging mechanism 16 as well as catheter body 18. Alternatively, an intermediate body 30 may include the charging mechanism. In addition, the fluid used in the balloon catheter system may be a gas or a liquid.

The preceding specific embodiments are illustrative of the practice of the invention. It is to be understood, therefore, that other expedients known to those skilled in the art or disclosed herein may be employed without departing from the invention or the scope of the claims. For example, the present invention further includes within its scope methods of making and using systems for transurethral ablation, as described herein.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1. A catheter system comprising:
a catheter defining an internal lumen;
a balloon mounted on the catheter, the balloon defining an internal chamber in fluid communication with the lumen;
a charging mechanism including a closed reservoir, a passage, and an actuator to charge and discharge the balloon with at least a portion of a volume of fluid contained in the closed reservoir, wherein the closed reservoir is in fluid communication with the lumen via the passage and substantially sealed from an environment outside the catheter system; and
a pressure sensor in fluid communication with the lumen to sense a pressure of the fluid.

2. The catheter system of claim 1, wherein the fluid is a gas.

3. The catheter system of claim 1, wherein the fluid is a liquid.

4. The catheter system of claim 1, further comprising a monitoring device to monitor the sensed pressure.

5. The catheter system of claim 1, further comprising a sensor body that houses the sensor, and a catheter body coupled to a proximal end of the catheter and the sensor body, the catheter body defining a channel for fluid communication between the lumen and the sensor, wherein the reservoir and the charging mechanism are housed in the sensor body.

6. The catheter system of claim 5, further comprising a seal member disposed between the sensor body and the catheter body.

7. The catheter system of claim 6, wherein the seal member includes a deformable surface that deforms under compression upon engagement of the sensor body and the catheter body to produce a fluid seal about the channel.

8. The catheter system of claim 1, further comprising a catheter body coupled to a proximal end of the catheter, wherein the reservoir and the charging mechanism are housed in the catheter body.

9. The catheter system of claim 8, further comprising a sensor body that houses the sensor, wherein the sensor body is coupled to the catheter body, wherein the catheter body defines a channel for fluid communication between the lumen and the sensor.

10. The catheter system of claim 9, further comprising a seal member disposed between the sensor body and the catheter body.

11. The catheter system of claim 10, wherein the seal member includes a deformable surface that deforms under compression upon engagement of the sensor body and the catheter body to produce a fluid seal about the channel.

12. The catheter system of claim 1, further comprising a sensor body that houses the sensor, a catheter body coupled to a proximal end of the catheter, and an intermediate body coupled to the sensor body and the catheter body, the catheter body defining a first channel for fluid communication with the lumen, the intermediate body defining a second channel for fluid communication between the first channel and the sensor, wherein the reservoir and the charging mechanism are housed in the intermediate body.

13. The catheter system of claim 12, further comprising a first seal member disposed between the intermediate body and the catheter body, and second seal member disposed between the sensor body and the intermediate body.

14. The catheter system of claim 13, wherein the first seal member includes a deformable surface that deforms under compression upon engagement of the sensor body and the intermediate body to produce a fluid seal about one of the first channel and the second channel.

15. The catheter system of claim 1, wherein the actuator includes a piston, and the distal end of the piston is movable to drive at least a portion of the fluid out of the reservoir and into the lumen to charge the balloon.

16. The catheter system of claim 1, wherein the actuator includes a screw, wherein the distal end of the screw is movable to drive at least a portion of the fluid out of the reservoir and into the lumen to charge the balloon.

17. The catheter system of claim 1, wherein the charging mechanism includes an elastic nipple, wherein pushing the nipple charges the balloon.

18. The catheter system of claim 1, wherein the pressure sensor comprises a strain gauge.

19. A method comprising:
placing a catheter into a patient, wherein the catheter includes an internal lumen and a balloon defining an interior chamber in fluid communication with the lumen;
sensing a first pressure within the lumen;
charging the balloon with at least a portion of a volume of fluid contained within a closed reservoir; and
sensing a second pressure within the lumen.

20. The method of claim 19, wherein the fluid is a gas.

21. The method of claim 19, wherein the fluid is a liquid.

22. The method of claim 19, wherein sensing a first pressure of the fluid within the closed reservoir comprises calibrating the catheter system such that the pressure within the catheter system is approximately zero.

23. The method of claim 19, further comprising discharging the balloon.

24. The method of claim 19, further comprising removing the catheter from the patient.

25. The method of claim 23, further comprising recharging the balloon.

26. The method of claim 19, wherein charging the balloon includes pushing on an elastic nipple.

27. The method of claim 19, wherein charging the balloon includes moving an actuator into the closed reservoir.

28. The method of claim 27, wherein the actuator is one of a piston and a screw.

29. The method of claim 23, wherein discharging the balloon comprises venting the fluid via an opening in the catheter system.

30. The method of claim 23, wherein discharging the balloon comprises removing an actuator from the closed reservoir of fluid, thereby allowing the volume of the closed reservoir to increase.

31. A catheter system comprising:
a catheter defining an internal lumen;
a balloon mounted on the catheter, the balloon defining an internal chamber in fluid communication with the lumen;
means for charging the balloon with at least a portion of a volume of fluid contained within a closed reservoir, wherein the reservoir is substantially sealed from an environment outside the catheter system; and
means for sensing a pressure of the fluid.

32. The catheter system of claim 31, further comprising a monitoring device to monitor the sensed pressure.

33. The catheter system of claim 31, further comprising a sensor body that houses the sensing means, and a catheter body coupled to a proximal end of the catheter and the sensor body, the catheter body defining a channel for fluid communication between the lumen and the sensor, wherein the reservoir and the charging means are housed in the sensor body.

34. The catheter system of claim 31, further comprising a catheter body coupled to a proximal end of the catheter, wherein the reservoir and the charging means are housed in the catheter body.

35. The catheter system of claim 31, further comprising a sensor body that houses the sensor, a catheter body coupled to a proximal end of the catheter, and an intermediate body coupled to the sensor body and the catheter body, the catheter body defining a first channel for fluid communication with the lumen, the intermediate body defining a second channel for fluid communication between the first channel and the sensor, wherein the reservoir and the charging means are housed in the intermediate body.

36. A catheter body for a balloon catheter system, the catheter body comprising:
a first fitting to couple the catheter body to a catheter;
a second fitting to couple the catheter body to a sensor body;
a charging mechanism including a closed reservoir, a passage, and an actuator to charge and discharge a balloon mounted on the catheter via a lumen with at least a portion of a volume of fluid contained in the closed reservoir, wherein the closed reservoir is in fluid communication with the lumen via the passage and substantially sealed from an environment outside the balloon catheter system.

37. A sensor body for a balloon catheter system, the sensor body comprising:
a first fitting to couple the sensor body to a catheter body;
a second fitting to couple the sensor body to a monitor;
a charging mechanism including a closed reservoir, a passage, and an actuator to charge and discharge a balloon mounted on the catheter via a lumen with at least a portion of a volume of fluid contained in the closed reservoir, wherein the closed reservoir is in fluid communication with the lumen via the passage and substantially sealed from an environment outside the balloon catheter system.

38. An intermediate charging body for a balloon catheter system, the charging body comprising:
a first fitting to couple the charging body to a catheter body coupled to a balloon catheter;
a second fitting to couple the charging body to a sensor body;
a charging mechanism including a closed reservoir, a passage, and an actuator to charge and discharge a balloon mounted on the catheter via a lumen with at least a portion of a volume of fluid contained in the closed reservoir, wherein the closed reservoir is in fluid communication with the lumen via the passage and substantially sealed from an environment outside the balloon catheter system.
